(19) United States
(12) Patent Application Publication
(10) Pub. No.: US 2019/0150773 A1
(43) Pub. Date: May 23, 2019

(54) CONTINUOUS CARDIAC MONITORING AND REAL TIME EPISODE DETECTION SYSTEM

(71) Applicant: CARDIAC DESIGN LABS PRIVATE LIMITED, Bangalore (IN)

(72) Inventor: Anand Madanagopal, Bangalore (IN)

(21) Appl. No.: 16/095,948

(22) PCT Filed: Mar. 10, 2017
(86) PCT No.: PCT/IN2017/050089

§ 371 Date: Oct. 24, 2018

(30) Foreign Application Priority Data
Apr. 27, 2016 (IN) 201641014618

(51) Int. Cl. A61B 5/0472; A61B 5/00; A61B 5/044; A61B 5/0408; A61B 5/0468

(52) U.S. Cl. CPC A61B 5/0472; A61B 5/746; A61B 5/044; A61B 5/7282; A61B 5/7405; A61B 5/04085; A61B 2562/222; A61B 5/4884; A61B 5/7264; A61B 5/6801; A61B 2562/04; A61B 2560/0431; A61B 5/0468

(57) ABSTRACT

A system and method for cardiac monitoring, which provides real time analysis, is enclosed. The system includes a memory unit, a processor that executes the set of modules. The set of modules includes a live cardiac monitor module 302, a clinical analysis module 306, a ST analysis display module 308, and an episode categorization and display module 308. The live cardiac monitor module 302 may continuously monitor and display the current condition of the patient 102 during an ECG diagnosis. In another embodiment, the clinical analysis module 306 simultaneously conducts a real-time arrhythmia analysis and a ST analysis on a same set of data during an ECG monitoring to detect episodes. The episode categorization and display module 310 categorizes and displays the detected episodes of the patient 102 in different colors that indicate a level of severity of each detected episodes.

### BACKGROUND

### Technical Field

The embodiments herein generally relate to a cardiac monitoring system, and more particularly, to a body worn unit, a base unit, remote unit, and a server for continuous cardiac monitoring and real time episode detection.

### Description of the Related Art

Cardiac disease is one of the most leading causes for deaths throughout the globe. Numerous studies have shown that early detection is critical for survival. One of the most fundamental methods for diagnosing cardiac conditions is Electrocardiogram (ECG) recording. The primary instrument used for ECG diagnostics is the resting ECG. This typically captures electrical activity of the heart for a duration of 10 to 12 seconds. The condition has to be persistent during the capture of the ECG. Therefore the resting ECG is only good for detecting acute episodes typically used for symptoms like chest pain and symptoms like fatigue, breathing problems, and dizziness. The resting ECG can be used only when the patient is in a resting position, and is not ambulatory.

There are no frontline devices for diagnosing Arrhythmia and Coronary Artery diseases, other than the Resting ECG in small hospitals. The 10-second resting ECG is an inadequate tool to diagnose the above conditions early and effectively and cases often go undiagnosed. Both these conditions require the ECG to be taken during ambulatory mode continuously for comprehensive analysis. The ECG devices used in ambulances are 12 Lead Resting ECGs and they have difficulty when in motion. Some such devices use telemetry. Multiple resting ECGs need to be taken en route in the ambulance to establish the stage of the patient's condition.

A Holter monitor is a mobile device for monitoring the ECG for longer durations of time such as for more than 1 day. It is useful for observing cardiac arrhythmias, which would be difficult to identify in a shorter period of time. The current Holter and event monitoring devices use technical team to filter ECGs and bring out the episodes. Some drawbacks of this diagnostic approach are that it is expensive and time consuming, analysis is done post monitoring, and remote monitoring of patients cannot be effective.

For patients having more transient symptoms, a cardiac event monitor that can be worn for longer durations say few days to week or for a month. The event monitor is typically smaller and sleeker than the Holter. In some cases the patient triggers the event monitor, so in case the patient fails to turn the recorder on, some arrhythmias that do not cause obvious symptoms may not be detected. In other cases the ECG gets recorded or uploaded regularly or when some changes are seen. A backend team is used for reading through the multiple ECG to bring about the episodes for review.

In Coronary Care Units (CCUs) or Intensive Care Units (ICUs), patient monitors are used for Continuous rhythm analysis. However, if comprehensive data is needed then multiple resting ECGs have to be taken. Sometimes Holters are used to generate more data but nothing comprehensive and real-time is used today.

A treadmill test may be conducted to detect an ischemic condition based on an ST segment. The treadmill test involves inducing stress on the patient and noting changes in the ST segment. The Holter monitor, the event monitor, and the treadmill test can typically be conducted only at large hospitals, and have to be administered and used by cardiologists.

Accordingly, there remains a need for comprehensive real time cardiac monitoring to detect an early onset of coronary artery disease without requiring the patient to visit a large hospital, and without requiring the presence of a cardiologist for administering the equipment.

### SUMMARY

In view of the foregoing, an embodiment herein provides A continuous ECG monitoring and real time episode detection system that consists of a body worn device that is worn by a patient that collects eight channel ECG signals through a cable that comprises ten electrodes fixed on the patient. A base unit that is a single device that performs functions of a Holter, a resting ECG, and an event monitor and stress test, that consists of a base unit processor that executes a set of base unit modules that display, record, process, and clinically analyse live ECG data received from the body worn device. The base unit modules consists of a live ECG module implemented on the base unit processor that continuously displays a twelve lead ambulatory ECG on a live monitor screen, a clinical analysis module implemented on the base unit processor that conducts a real-time arrhythmia analysis and a ST analysis on a same set of the twelve lead ambulatory ECG for a short term monitoring or a long term monitoring to detect episodes, a ST analysis display module implemented on the base unit processor that determines an average graph for each ECG lead of the twelve lead ambulatory ECG and displays the average graph on the live monitor screen, and an episode categorization module implemented on the base unit processor that categorizes and displays the detected episodes of the patient in different colours in the form of a timeline, depending on a level of severity of the detected episodes.

In one embodiment, the base unit consists ofa diagnosis mode selection module implemented on the base unit processor that processes a selection of a mode of diagnosis selected from the ambulatory (Holter) mode, the resting ECG mode, or the stress test mode, an ambulatory mode module implemented on the base unit processor that continuously monitors the patient for a long term or a short term monitoring, a resting ECG mode module implemented on the base unit processor that analyses the patient for a fixed duration and immediately raises alerts if an episode is detected, and a stress test mode module implemented on the base unit processor that implements a clinical stress protocol when the patient is running on a treadmill. A live cardiac monitor module implemented on the base unit processor that continuously monitors the patient and displays the ECG.

In another embodiment, the continuous ECG monitoring and real time episode detection system consists of a remote unit, the remote unit consists of a remote unit processor that executes a set of remote unit modules. The remote unit modules consists of a connection status module implemented on the remote unit processor that indicates connection status between the remote unit and the base unit, a live ECG module implemented on the remote unit processor that displays the ECG from the base unit, when a request is made, a categorized episode timeline module implemented on the remote unit processor that displays various episodes detected and has a filter option that filters for viewing a selected episode, a referral module implemented on the remote unit processor that allows a doctor to refer a diagnosis or snapshots of the patient to other doctors. The doctor may select another doctor by using an auto referral option that transfers the diagnosis and the snapshots of the patient to the another doctor and a ECG parameters module displays (i) a QTC interval, (ii) a QT interval, (iii) a RR interval, and (iv) a PR interval.

In yet another embodiment, the body worn device consists of an lightweight one millimeter thin coaxial cable, a very small connector, a power LED that indicates the body worn device battery status, a connectivity LED that indicates a connection status with the base unit, a patient triggered button that is pressed by the patient during an uneasy situation and an episode will be generated in the base unit and will be uploaded and received by the remote unit, and a buzzer that beeps whenever there are disruptions in connectivity with the base unit and when there is a lead-off.

In yet another embodiment, the clinical analysis module consists of a noise detection and PQRST identification module implemented on the base unit processor that detects noise based on noise threshold levels and analysis of each beat, where segments are divided between one R-peak to another R-peak looking for high and low frequency components, and a signal is logically parsed based on a amount of segments of a wave, determining where wave deviation occurs, a reverse correction and beat selection module implemented on the base unit processor that uses features and cross referencing on other leads for better beat selection, and beat characteristics are determined after a beat is detected and features of the beat are determined, a beat selection for ST segment display module implemented on the base unit processor that selects the beat based on the ST segment.

In yet another embodiment, the remote unit consists of a controlled alert configuration module implemented on the remote unit processor that enables the doctor to select a type of alarm notification for receiving alerts, an alert display module implemented on the remote unit processor that displays alerts assigned to the doctor, an annotation module implemented on the remote unit processor that allows the doctor to annotate or add comments regarding the ECG diagnosis on which an notification will be received on the base unit and will be available to view and will be available on the report generated for printing, and a report append module implemented on the remote unit processor that adds the snapshots of the diagnosis taken to the report.

In one aspect, a method of continuous cardiac monitoring and real time episode detection that obtains live ECG data from a body worn device attached to a patient. The method consists of collecting eight channel ECG signals through a cable that comprises ten electrodes fixed on the patient body, selecting a mode of diagnosis from at least one of (i) an ambulatory mode selection, (ii) a resting ECG mode selection, or (iii) a stress test mode selection, wherein the ambulatory mode monitors a patient for a long time period, the resting ECG mode monitors ECG for a fixed duration and immediately raises alerts when an episode is detected, and the stress test mode implements a clinical stress protocol when the patient is running on a treadmill during an ECG monitoring, continuously monitoring the patient during an ECG diagnosis and displaying the ECG of the patient, conducting a real time arrhythmia analysis and a ST analysis on a same set of data during the ECG monitoring to detect episodes, displaying a twelve lead ambulatory ECG on a live monitor screen, determining an average graph for all the ECG leads and displaying the average graph on a live monitor screen during ST Analysis mode, and categorizing and displaying detected episodes of the patient in different colors that indicate a level of severity of each the detected episodes.

In one embodiment, allowing a doctor to enable or disable at a base unit, (i) a system beep, (ii) an alarm beep, or (iii) an alarm popup and allowing the doctor to enable or disable at the base unit, (a) detection of the alarm conditions, (b) setting a time for periodic episode, of (c) setting a periodic count generation.

In another embodiment, capturing one or more snapshots at the base unit, during the ECG monitoring and adding the one or more snapshots at the base unit, to a patient report.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a overall system view consists of a base unit that obtains live ECG data from a body worn device attached to a patient and communicates with a server and a remote unit through a network according to an embodiment herein;

FIG. 2A illustrates a perspective view of the body worn device of FIG. 1 according to an embodiment herein;

FIG. 2B illustrates a block diagram of the body worn device of FIG. 1 according to an embodiment herein;

FIG. 3 illustrates an exploded view of the base unit of FIG. 1 according to an embodiment herein;

FIG. 4 illustrates an exploded view of a clinical analysis module in the base unit of FIG. 2 according to an embodiment herein;

FIG. 5 illustrates an exploded view of the remote unit of FIG. 1 according to an embodiment herein;

FIG. 6 illustrates an exploded view of the server of FIG. 1 according to an embodiment herein;

FIG. 7 illustrates a user interface view of a live cardiac monitor module in the base unit of FIG. 1 according to an embodiment herein;

FIG. 8 illustrates a user interface view of a diagnosis module in the base unit of FIG. 1 according to an embodiment herein;

FIG. 9 illustrates a user interface view of a diagnosis settings module displaying the various settings available in the base unit of FIG. 1 according to an embodiment herein;

FIG. 10 illustrates a user interface view of an episode categorization and display module in the base unit of FIG. 1 according to an embodiment herein;

FIG. 11 illustrates a user interface view of a ST analysis display module in the base unit of FIG. 1 according to an embodiment herein;

FIG. 12 illustrates a user interface view of an attendant report module in the base unit of FIG. 1 according to an embodiment herein;

FIG. 13 illustrates a user interface view of an assigned diagnosis module in the remote unit of FIG. 1 according to an embodiment herein;

FIG. 14 illustrates a user interface view of an auto referral option in the remote unit of FIG. 1 according to an embodiment herein;

FIG. 15 illustrates a user interface view of diagnosis summary module in the remote unit of FIG. 1 according to an embodiment herein;

FIG. 16 illustrates a user interface view of an alert configuration module in the remote unit of FIG. 1 according to an embodiment herein;

FIG. 17 illustrates a user interface view of an alert display module in the remote unit of FIG. 1 according to an embodiment herein;

FIG. 18 illustrates a user interface view of a categorized episode timeline module in the remote unit of FIG. 1 according to an embodiment herein;

FIG. 19 illustrates a user interface view of an annotation module in the remote unit of FIG. 1 according to an embodiment herein;

FIG. 20 illustrates a user interface view of a ECG parameters module in the remote unit of FIG. 1 according to an embodiment herein;

FIG. 21 illustrates a user interface view that illustrates filtering snapshots added to a report in the remote unit of FIG. 1 according to an embodiment herein;

FIG. 22 illustrates a user interface view that illustrates a referral module in the remote unit of FIG. 1 according to an embodiment herein;

FIG. 23 illustrates a user interface view of a report generation module in the remote unit of FIG. 1 according to an embodiment herein;

FIG. 24 is a flow diagram illustrating a method of continuous cardiac monitoring and real time episode detection that obtains live ECG data from the body worn device attached to the patient of FIG. 1 according to an embodiment herein;

FIG. 25 illustrates an exploded view of the base unit or the remote unit of FIG. 1 according to an embodiment herein; and

FIG. 26 illustrates a schematic diagram of a computer architecture of the base unit, the remote unit, or the server of FIG. 1 used according to the embodiments herein.

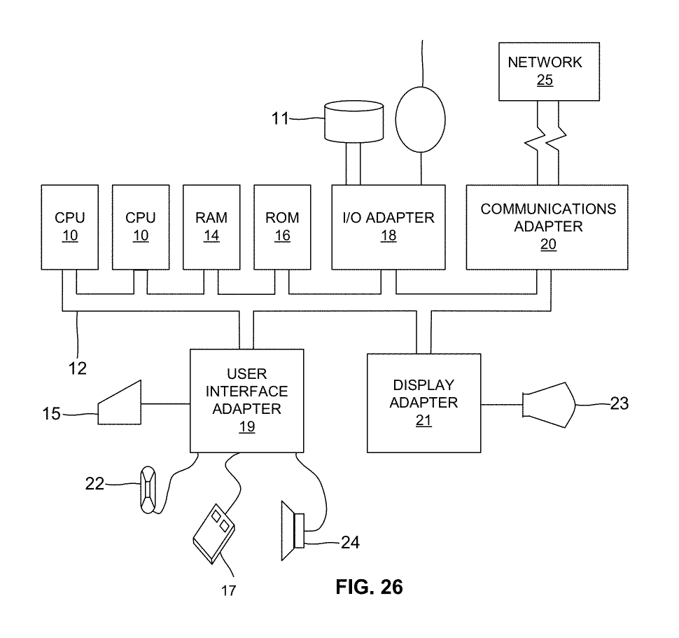

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In an embodiment, the continuous cardiac monitoring system includes a body worn device, a base unit, a server and a remote unit.

In an embodiment, a clinical analysis is done in real time in the base unit based on ECG signals acquired in the body worn device, and episodes are detected and reported to a remote unit as alerts along with clinical analysis information.

In an embodiment, the patient is able to report an episode in case of discomfort, due to which an alert mechanism is activated, sending an ECG snap shot of the episode to a base unit, server or control unit and a remote device. The patient can trigger an event using a patient triggered button on the body worn device in case of discomfort and event is processed and recorded in base unit and then sent to the server and then further sent to remote application.

In an embodiment, diagnosis of an ECG is made by a method of finding P, Q, R, S, T points on the ECG graph, recording each beat; analyzing each beat on the parameters and features pertaining to height and shape; accessing noise; marking high frequency and low frequency noise sections; mapping a beat shape; performing reverse correction of the beat selected and type of the beat is captured; and re analyzing the selection of beat.

In an embodiment, diagnosis is made by a clinical analysis module in the base unit, and categorized as a normal ECG, patient reported event, and/or an episode detection, and displayed as categorized on the UI of the base unit and the remote device. Any anomaly in the ECG is recorded and thus reported. Various cardiac conditions like arrhythmia, ischemia, are diagnosed in the base unit and reported to the remote unit.

In an embodiment, ST segment analysis is performed to extract information on the ischemia condition of the heart.

In an embodiment, a diagnostic history, activity of the patient, and a status of the body worn device are displayed on a remote device.

In an embodiment, ECG and metrics data of a relevant episode or sample may be fetched by the remote unit for comprehensive diagnosis.

In an embodiment, the body worn device is lightweight, convenient with 12 leads and may be used for a longer period of monitoring, while the patient is able to carry out routine activity.

### DETAILED DESCRIPTION OF DRAWINGS

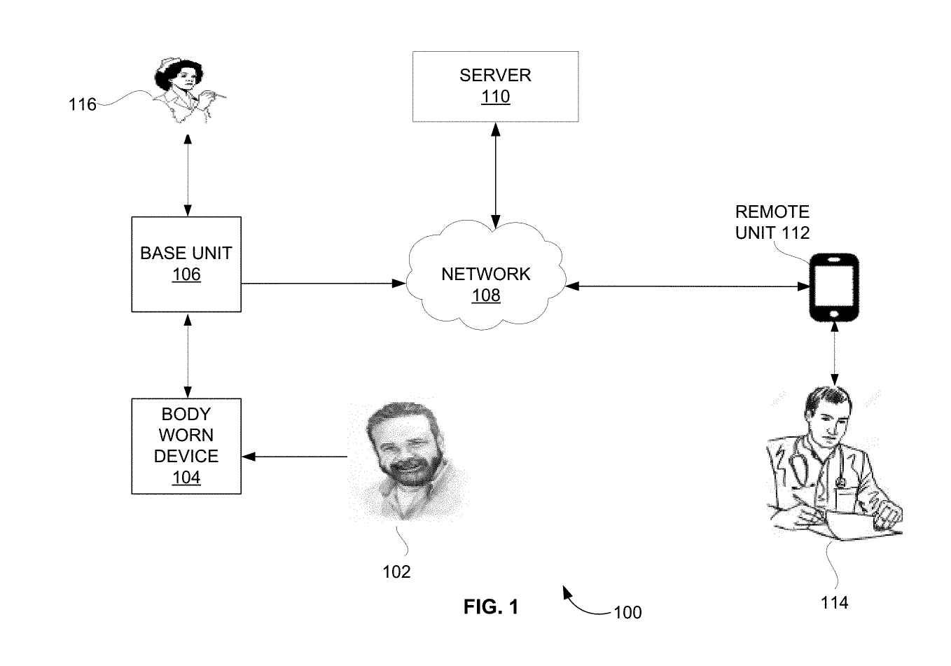

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. Referring now to the drawings, and more particularly to FIGS. 1 through 12, where similar reference characters denote corresponding features consistently throughout the figures, there are shown preferred embodiments.

FIG. 1 illustrates a overall system view 100 which consists of a base unit 106 that obtains live ECG data from a body worn device 104 attached to a patient 102 and communicates with a server 110 and a remote unit 112 through a network 108 according to an embodiment herein. The system view 100 includes a patient 102, a body worn device 104, a base unit 106, a network 108, a server 110, a remote unit 112, a doctor 114, and an attendant 116. The attendant 116 may be a nurse or a technician, in one embodiment. The body worn device 104 is worn by the patient 102 throughout as the diagnosis occurs over a long period of time. The base unit 106 receives ECG data from the body worn device 104, records, processes, and analyzes the data clinically, and communicates with the remote unit 112 and the server 110 through the network 108.

In one embodiment, the body worn device 104 is a lightweight device that collects eight channel ECG signals through 10 electrodes fixed on the body of the patient 102 at the required positions. In one embodiment, there are a total of 10 electrodes out of which four of them are limb electrodes and six of them are chest electrodes. In another embodiment, the body worn device 104 also includes of a first sensor component and a second sensor component. The first sensor component may include electrodes for ECG pickup and the second sensor component may include an accelerometer for the movement pickup of the patient 102. The accelerometer is useful in detecting various patient activities like stationary, walking, standing, and running.

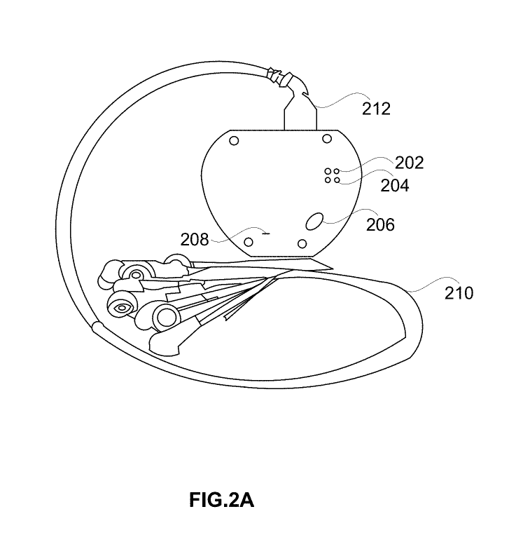

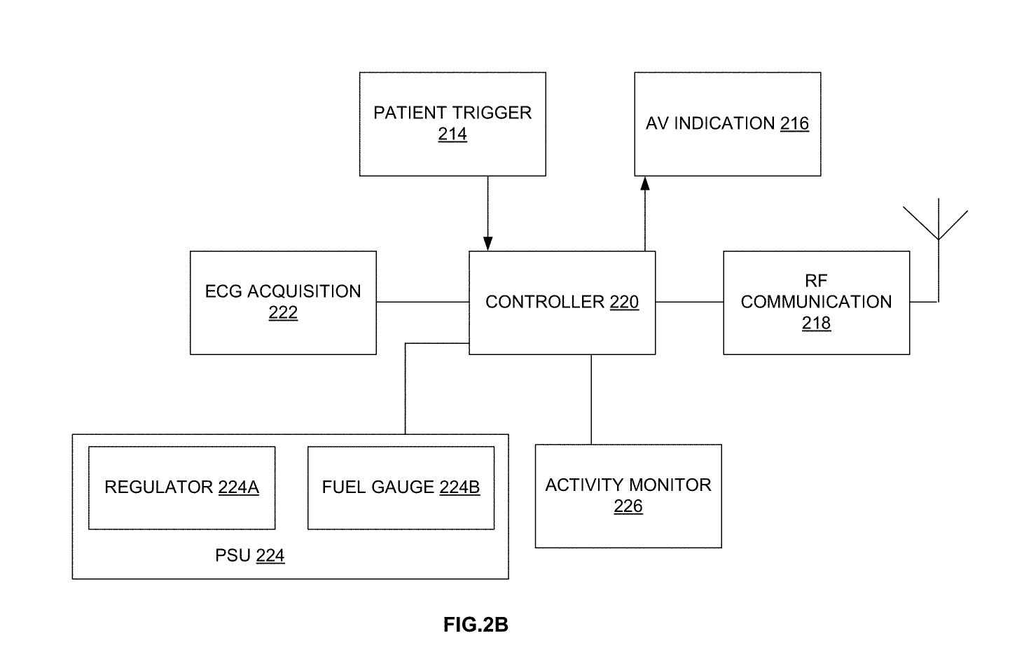

FIG. 2A illustrates a perspective view 200 of the body worn device 104 of FIG. 1 according to an embodiment herein. The perspective view 200 includes a power LED 202, a connectivity LED 204, a Patient triggered button 206, a buzzer 208, and a plurality of ECG electrodes 210. The power LED 202 indicates whether the body worn device 104 is on and functioning. The connectivity LED 204 indicates the status of a blue tooth connection with the base unit 106. The Patient triggered button 206 is provided for the patient 102 to press whenever he/she feels uneasy and an episode will be generated in the base unit and will be uploaded and received by the remote unit. The buzzer 208 may beep whenever there are any disruptions in connectivity and when there is a lead-off. The plurality of ECG electrodes 210 are fixed on the patient 102 chest at appropriate positions using fine and light ECG cables to detect the ECG signals during the ECG diagnosis.

FIG. 2B illustrates a block diagram 200 of the body worn device 104 of FIG. 1 according to an embodiment herein. The block diagram 200 includes a patient trigger 214, an AV indication 216, a RF communication unit 218, a controller 220, an ECG acquisition 222, a PSU 224, and an activity monitor 226. The patient trigger 214 acts as an input and may be triggered whenever a patient 102 feels uncomfortable. The AV indication 216 indicates the status of the battery and the status of the connectivity between the body worn device 104 and the base unit 106. In one embodiment, a power LED is used to indicate the battery status. In one embodiment, when the power LED is slow blinking green, then that indicates that the battery has charge and when the power LED is blinking red, then it indicates that the battery is low in charge. In another embodiment, a communication status LED is used to indicate the connectivity status. In one embodiment, when the communication status LED is fast blinking green, then it indicates that the base unit 106 connection is established and data is being transmitted. When the communication status LED is blinking red, then it may indicate that there is no connection established.

The RF communication 218 unit may handle all the communication mechanisms. The controller 220 may handle all the handshaking operations of the RF communication 218. The ECG acquisition unit 222 includes 8 channels and picks up the analog signal from the body worn device 104, and converts it in to a digital signal using an internal Analog to digital convertor, according to one embodiment. The PSU 224 includes a regulator 224A and a fuel gauge 224B. In one embodiment, the regulator 224A generates the required operational voltage for the body worn device 106. In another embodiment, the fuel gauge 224B determines the percentage of charge available in the battery. The activity monitor 226 captures the patient 102 activities like resting, sleeping, and walking.

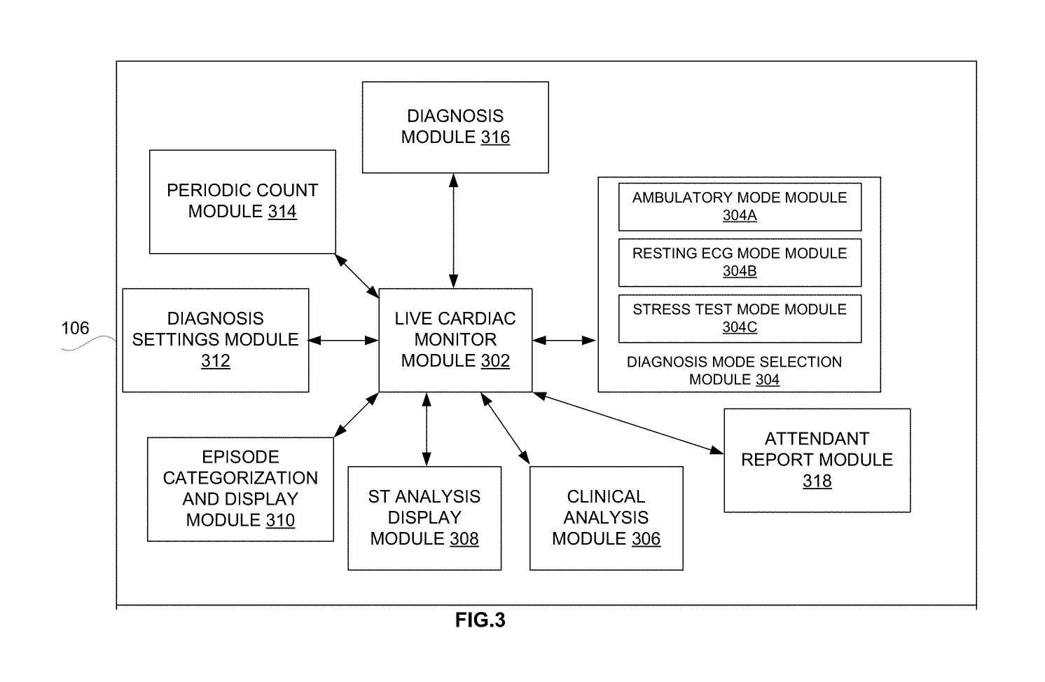

FIG. 3 illustrates an exploded view 300 of the base unit 106 of FIG. 1 according to an embodiment herein. The exploded view 300 includes a live cardiac monitor module 302, a diagnosis mode selection module 304, a clinical analysis module 306, a ST analysis display module 308, an episode categorization and display module 310, a diagnosis setting module 312, a periodic count module 314, a diagnosis module 316, and an attendant report module 318. The cardiac monitor module 302 continuously monitors the patient 102 during the ECG diagnosis for a long period of time. In one embodiment, the cardiac monitor module 302 displays two leads in the landscape mode and all the twelve leads in the portrait mode. The diagnosis mode selection module 304 processes a mode of selection of a mode of diagnosis. In one embodiment, the diagnosis mode selection module 304 consists of an ambulatory (Holter) mode module 304A, a resting ECG mode module 304B, and a stress test mode module 304C. The ambulatory mode module 304A continuously monitors the patient 102 for a long term duration. The resting ECG mode module 304B continuously monitors the patient 102 for a fixed time duration, and immediately raises an alert when an episode is detected. The stress test mode module 304C implements a clinical stress protocol in conjunction with a treadmill for the patient 102.

The clinical analysis module 306 conducts a real time arrhythmia analysis and a ST analysis on a same set of twelve lead ambulatory ECG for a short term monitoring or a long term monitoring. The ST analysis display module 308 displays an average graph of each lead of the twelve lead ambulatory ECG. In one embodiment, the ST analysis display module 308 shows the average graph of 16 seconds ECG for each of the twelve leads and also displays a ST deviation value.

The episode categorization and display module 310 displays the detected episodes of the patient 102 in different colors in the form of a timeline. The diagnosis settings module 312 may include system settings, diagnostic settings, and advanced settings. In one embodiment, the system settings allows the doctor 114 to enable or disable the system beep, alarm beep, and the alarm popup. In another embodiment, the diagnostic settings allows the doctor 114 to enable or disable detection of the alarm conditions, setting the time for periodic episode, and setting a periodic count generation. The periodic episode count module 314 displays problems detected during the ECG analysis of the patient 102. In one embodiment, a yellow color is displayed if the patient 102 has major problems, a blue color is displayed when the patient 102 triggers, a white color is displayed if the patient 102 has minor problems and check samples, and a red color is displayed if the patient 102 is critical.

The attendant report module 318 generates a report containing the selected episodes during the ECG monitoring along with a summary. In one embodiment, the report may be generated by any one near the patient 102 and may be printed out as well. In another embodiment, the report includes comments given by the doctor 114 regarding the ECG diagnosis and the episodes detected. The diagnosis viewed by the doctor 114 may be marked and annotated as viewed. The Diagnosis module 316 where patient details are entered at the start of the monitoring and enables viewing of previous patient 102 details and also allows previous patient details to be deleted if they are no longer required.

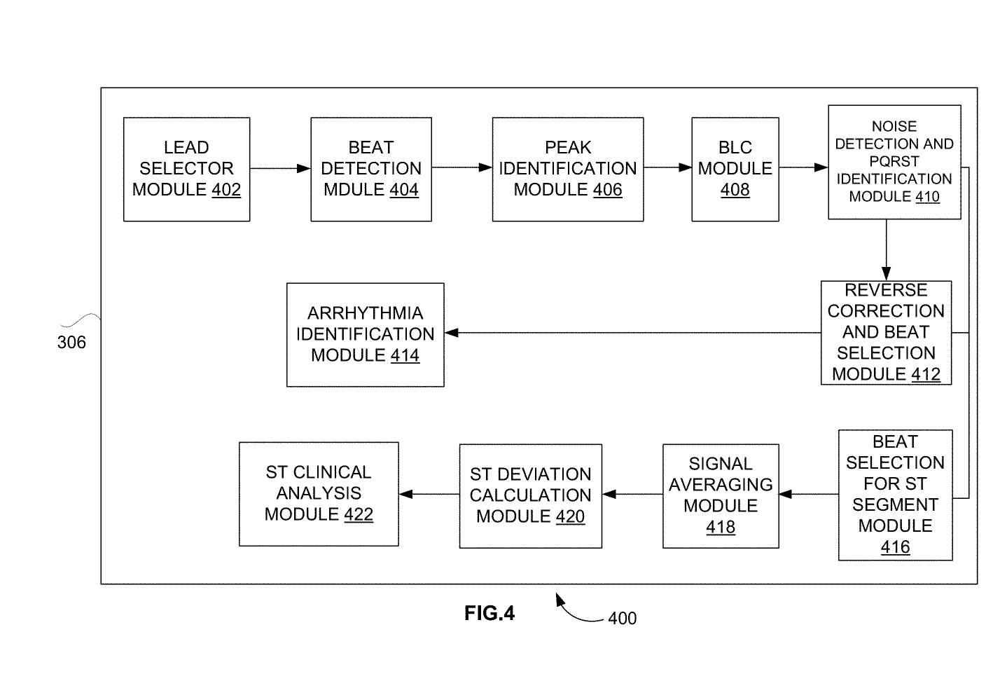

FIG. 4 illustrates an exploded view 400 of a clinical analysis module 304 in the base unit 106 of FIG. 2 according to an embodiment herein. The exploded view 400 includes a lead selector module 402, a beat detection module 404, a peak identification module 406, a BLC module 408, a noise detection and PQRTS identification module 410, a reverse correction and beat selection module 412, an arrhythmia identification module 414, a beat selection for ST segment display module 416, a signal averaging module 418, a ST deviation calculation module 420, and a ST clinical analysis module 422. The lead selector module 402 identifies the right lead based on lead noise and ECG parameters starting from the default lead. The beat detection module 404 feeds each sample to a QRS detector. In one embodiment, if a beat is detected, then a beat node will be created and will be added to a dynamic beat list, along with beat types and their rough R positions. The peak identification module 406 logically parses the identified beat to determine the exact QRS position. The BLC module 408 performs PQ knot detection for a base line correction.

The noise detection and PQRST identification module 410 is performed based on noise threshold levels and analyzing of each beat. In one embodiment, segments are divided between one R-peak to another R-peak looking for high and low frequency components, and threshold levels are used for detecting noises. In another embodiment, using R as reference the other points are gotten based on the segment of the wave, the signal is logically parsed to determine when wave deviation occurs and corresponding point is marked. The reverse correction and beat selection module 412 uses features and cross references another lead's beat selection to make corrections to the selection and type. In another embodiment, after a beat detection, its features are detected, representing beat characteristics. The arrhythmia identification module 414 identifies arrhythmia analysis based on a clinical rulebook, in one embodiment. The beat selection for ST Segment module 416 selects the beat based on the ST segment noise and other features from clinical rulebook. The signal averaging module 418 constructs signal average beats. The ST deviation calculation module 420 calculates J points and the ST clinical analysis module 422 calculates the ST segment and based on the clinical rulebook makes an outcome of the ischemia condition.

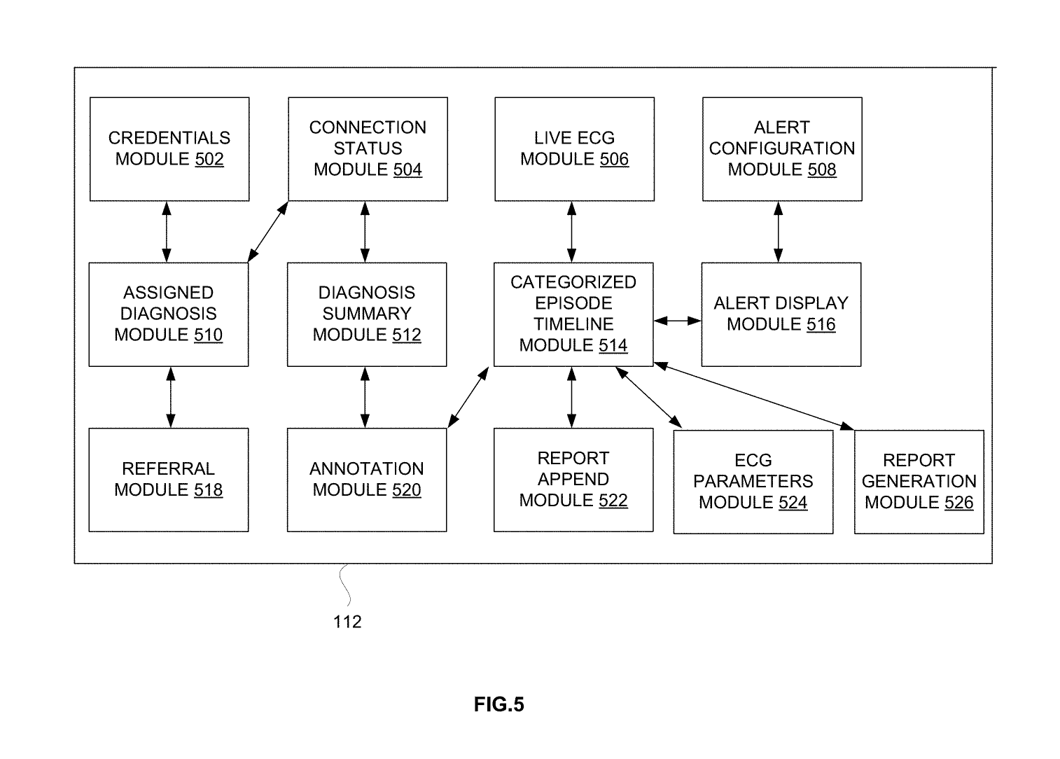

FIG. 5 illustrates an exploded view 500 of the remote unit 112 of FIG. 1 according to an embodiment herein. The exploded view 500 includes a credentials module 502, a connection status module 504, a live ECG module 506, an alert configuration module 508, an assigned diagnosis module 510, an diagnosis summary module 512, a categorized episode timeline module 514, an alert display module 516, a referral module 518, an annotation module 520, a report append module 522, a ECG parameters module 524, and a report generation module 526. The credentials module 502 enables the doctor 114 to login by entering his/her username and password. The connection status module 504 is an indicator showing whether the remote unit 102 is connected to the server.

The live ECG module 506 displays a live ECG to the doctor 112 on request, in one embodiment. The alert configuration module 508 enables a doctor to select the type of alarm notifications the doctor wants to receive. In one embodiment, the alert configuration module 508 interacts with the alert display module 516 so that the doctor 114 may filter the list by selecting the alarm type. The assigned diagnosis module 510 shows the list of diagnoses assigned to the doctor 114 as soon as he/she logs in. The diagnosis summary module 512 displays diagnosis details and the various episodes detected during the ECG diagnosis to the doctor 114. The categorized episode timeline module 514 displays the various episodes generated during the ECG diagnosis in a timeline format. In one embodiment, each snapshot generated is represented by colors where a yellow color indicates that the patient 102 has problems, a blue color indicates that the patient 102 has triggered, a white color indicates minor condition, and a red color indicates that the patient is critical.

The referral module 518 refers the diagnosis/snapshots taken during the ECG diagnosis to any other doctor 114. The annotation module 520 allows the doctor 114 to annotate or add his/her comments regarding the ECG diagnosis. The report append module 522 adds the snapshots to a report for printing. The ECG parameters module 524 enables the doctor to view ECG segments measurements pertaining to specific snapshot/episode of the patient 102. In one embodiment, the ECG parameters module 524 displays a QTC interval, a QT interval, a RR interval, and a PR interval. The report generation module 526 generates a report of a required diagnosis. The generated report will have the selected ECG snapshots with the patient 102 summary and details.

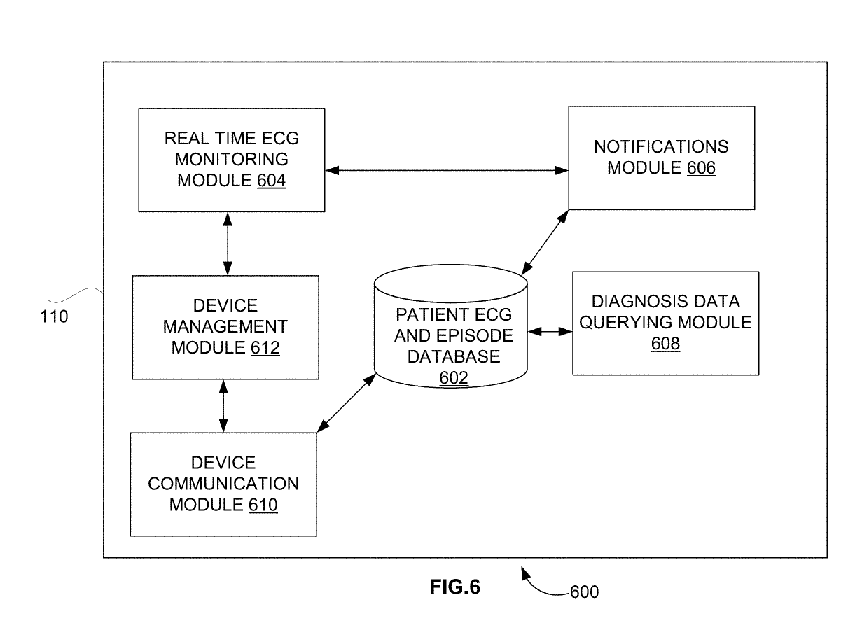

FIG. 6 illustrates an exploded view 600 of the server 110 of FIG. 1 according to an embodiment herein. The exploded view 600 includes a patient ECG and episode database 602, a real time ECG monitoring module 604, a notifications module 606, a diagnosis data querying module 608, a device communication module 610, and a device management module 612. The patient episode and ECG database 602 stores and organizes all the episode information of patients during ECG diagnosis. The real time ECG monitoring module 604 updates the doctor 114 regarding the current condition of the patient 102 during the ECG diagnosis. The notifications module 606 notifies or informs the users/doctor 114 when he/she is logged in, on the diagnoses which has been assigned to him/her and updates the doctor 114 of the episodes being generated. The diagnosis data querying module 608 queries the base unit 106 to fetch diagnosis data when requested by the remote unit 112, in one embodiment. The device communication module 610 enables communication between the base unit 106, and the remote unit 112, in one embodiment. The device management module 612 manages data for a plurality of base units 106 and a plurality of remote units 112 for multiple users (e.g., various health care centers, patients, doctors etc).

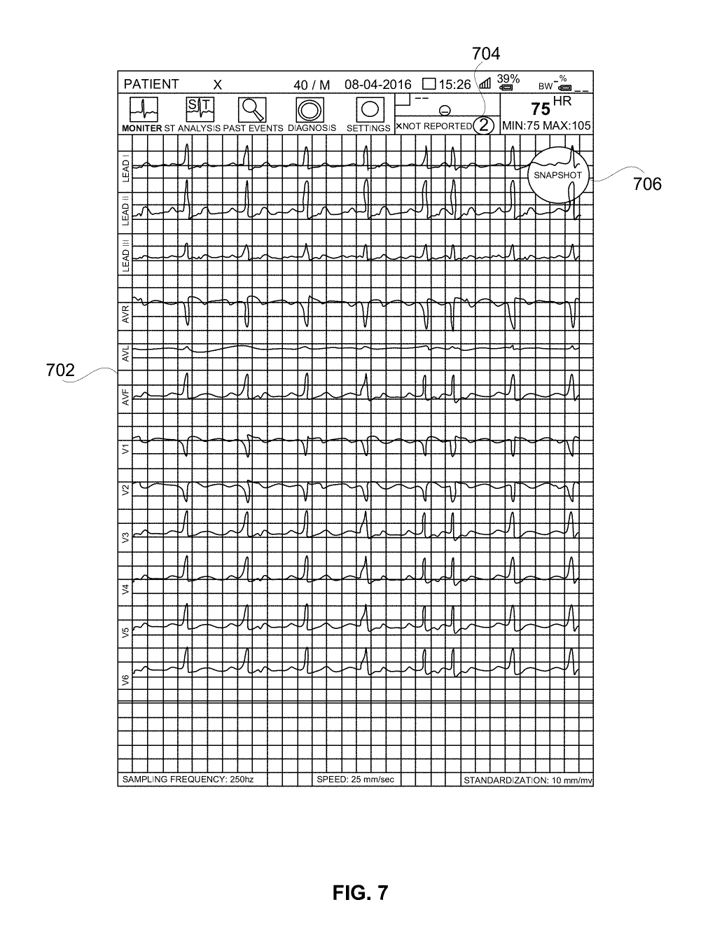

FIG. 7 illustrates a user interface view 700 of a live cardiac monitor module 302 in the base unit 106 of FIG. 1 according to an embodiment herein. The user interface view 700 includes a twelve ECG lead view 702, an alarm update 704, and a snapshot button 706. The lead view 702 displays two leads in the landscape mode and all the twelve leads in the portrait mode for the patient 102 during the ECG diagnosis. The alarm update 704 mentions the number of alarms generated and the time at which they were generated during the ECG diagnosis. The snapshot button 706 allows taking a 10 second snapshot of the required ECG during the patient 102 ECG diagnosis. Multiple snapshots may be captured during the ECG diagnosis.

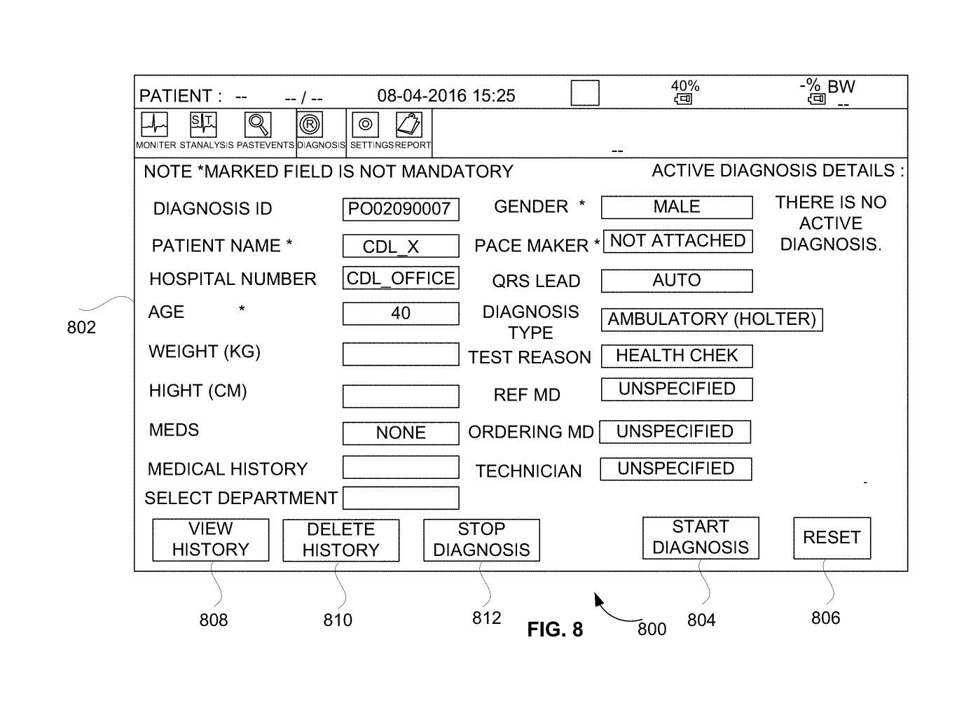

FIG. 8 illustrates a user interface view 800 a diagnosis module 316 in the base unit 106 of FIG. 1 according to an embodiment herein. The diagnosis module 318 is where the patient 102 details are captured and monitored. The user interface view 800 includes diagnosis details 802. The diagnosis details 802 may include information related to the patient diagnosis. In one embodiment, the diagnosis details 802 includes basic information regarding the patient 102 like a diagnosis ID, name of the patient 102, hospital name, age, gender, diagnosis type and reason for test. In another embodiment, the diagnosis details 802 includes a start diagnosis button 804 that is used for creating a diagnosis by filing in the patient details, and a stop diagnosis button 806 for stopping a currently running diagnosis. In yet another embodiment, the diagnosis details 802 further consists of a view history button 808 for viewing other diagnosis 102 diagnosis history, a delete history button 810 for deleting a particular diagnosis, and a reset button 812 for starting the entry again, which automatically removes previous entered information.

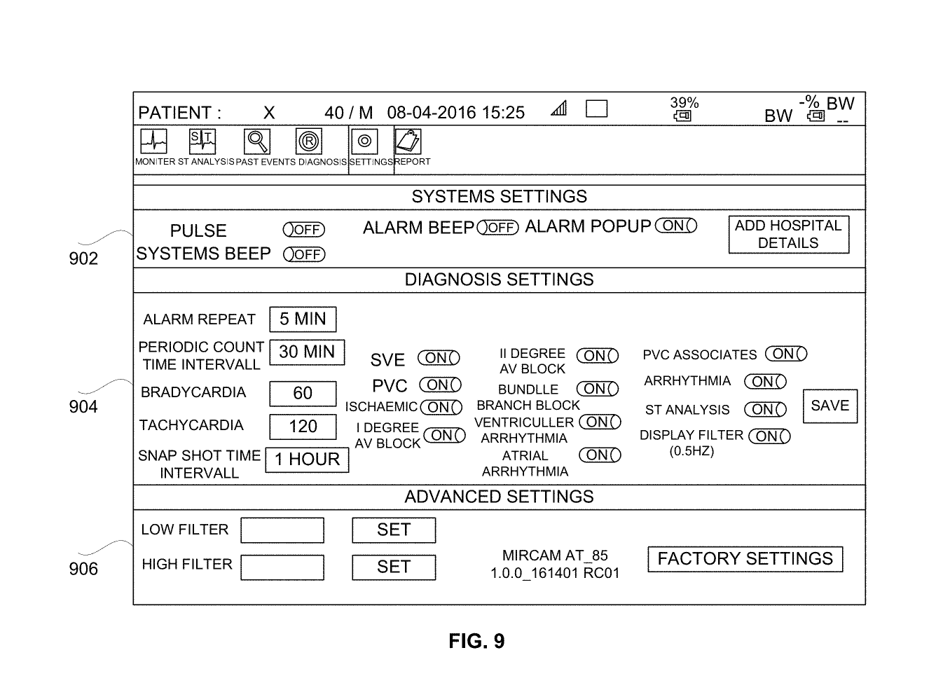

FIG. 9 illustrates a user interface view 900 of a settings page displaying the various settings available in the base unit 106 of FIG. 1 according to an embodiment herein. The user interface view 900 includes a system settings view 902, a diagnostic setting view 904, and an advanced settings view 906. The system settings view 902 allows and enables a system beep, an alarm beep, and an alarm popup, in one embodiment. The diagnostic settings view 904 allows the control of alarm detection, setting the time for periodic extension, and also setting the periodic count generation.

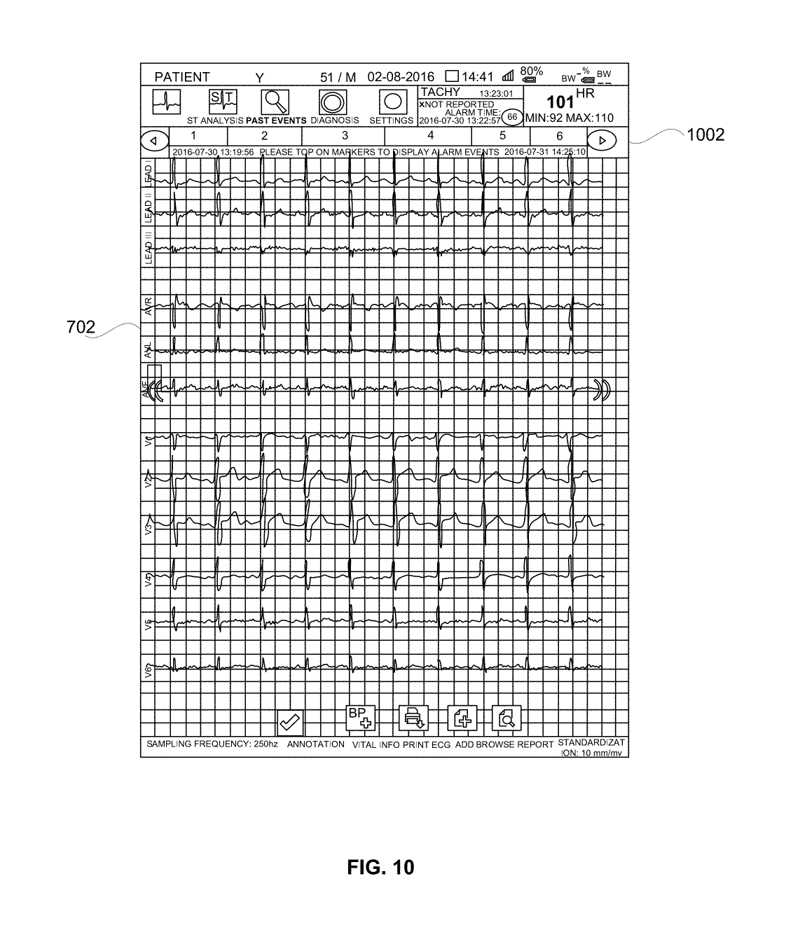

FIG. 10 illustrates a user interface view 1000 of an episode categorization and display module 308 in the base unit 106 of FIG. 1 according to an embodiment herein. The user interface view 1000 includes a color marker 1002. The color marker 1002 displays four colors that depict a type of alert. In one embodiment, a yellow color depicts that the patient 102 has major problems, a blue color indicates an episode that the patient 102 triggered, a white color for minor problems and check samples, and a red color indicates that the patient 102 is critical.

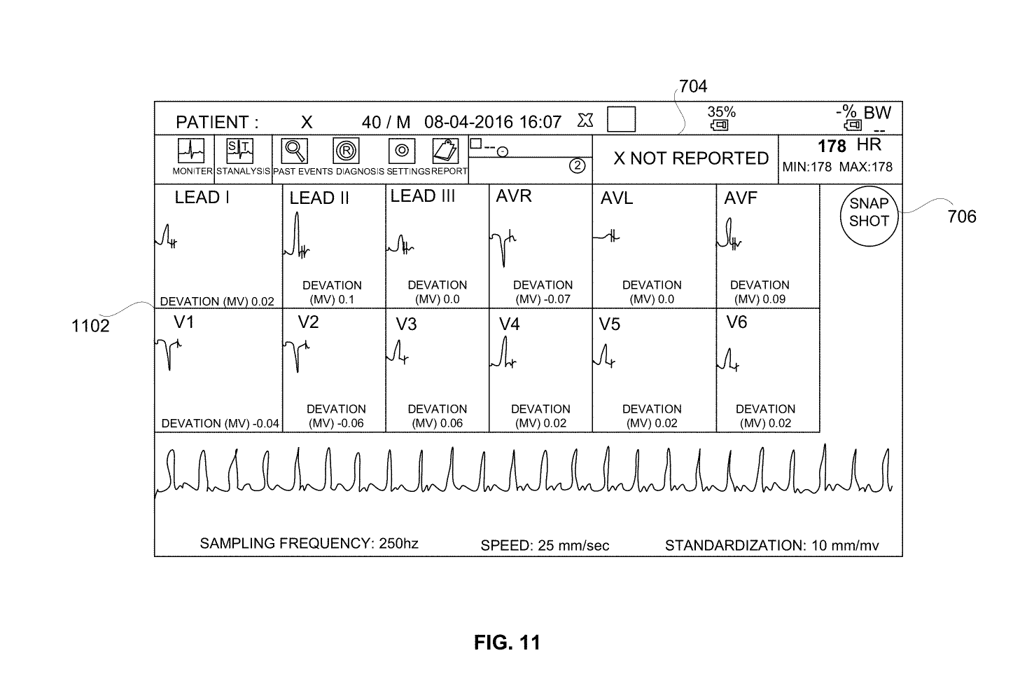

FIG. 11 illustrates a user interface view 1100 of the ST analysis display module 306 in the base unit 106 of FIG. 1 according to an embodiment herein. The user interface view 1100 includes a ST analysis page 1102 displaying the average graph of each lead. In one embodiment, the ST analysis page 1102 shows the average graph of 16 seconds ECG for each of the twelve leads and also displays a ST deviation value.

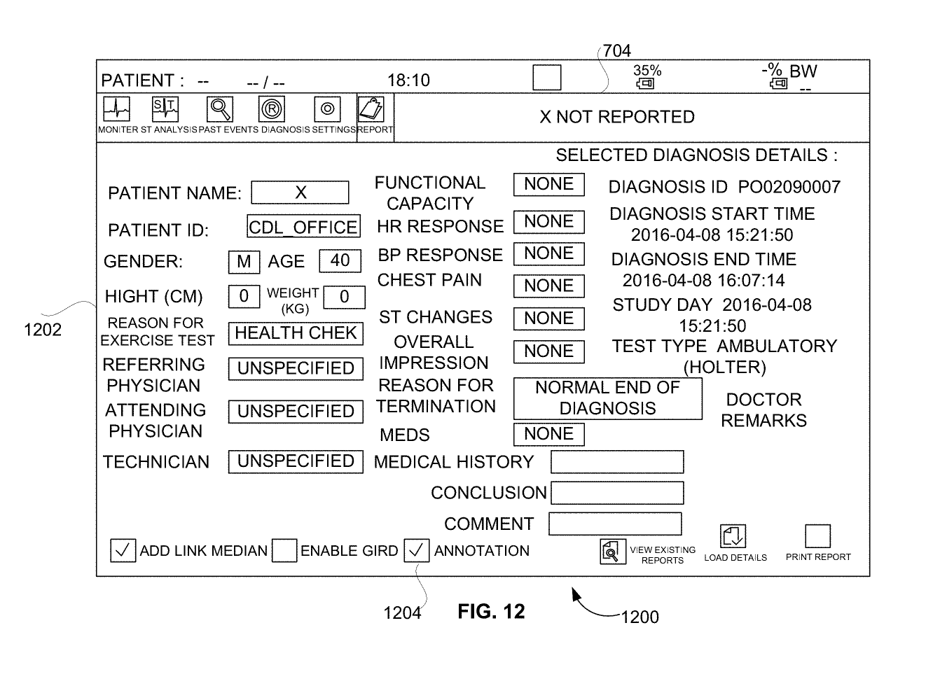

FIG. 12 illustrates a user interface view 1200 of an attendant report module 316 in the base unit 106 of FIG. 1 according to an embodiment herein. The user interface view 1200 includes a diagnosis report summary 1202. The diagnosis report summary 1202 includes the patient 102's basic information; and the doctor 112's comments regarding the diagnosis and episodes. An annotated button 1204 is ticked or un-ticked to indicate whether or not to include the annotation in the report.

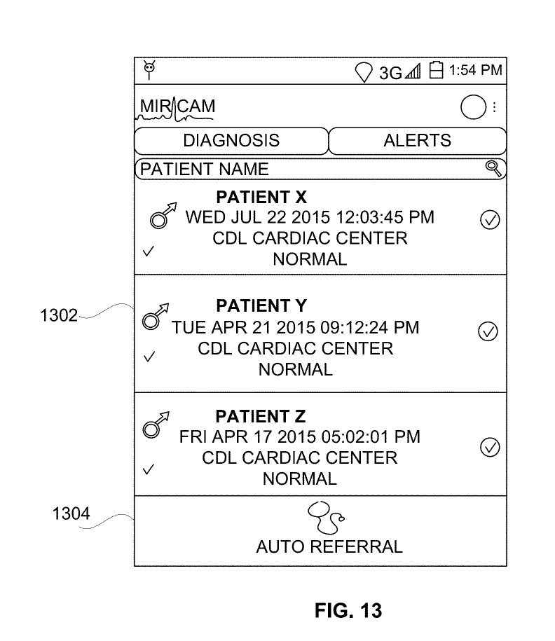

FIG. 13 illustrates a user interface view 1300 of an assigned diagnosis module 510 in the remote unit 112 of FIG. 1 according to an embodiment herein. The user interface view 1300 includes a list 1302 of diagnoses assigned to the doctor 112. The list 1302 displays the patient 102 name, unit name, diagnosis creation time, connection status, gender, and a diagnosis type, according to an embodiment.

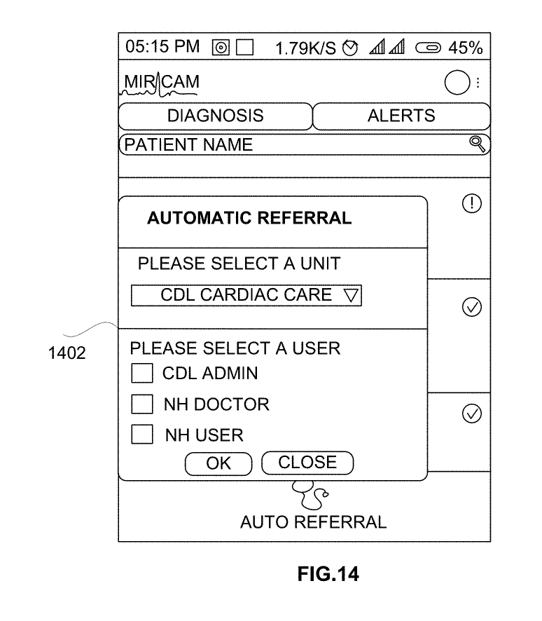

FIG. 14 illustrates a user interface view 1400 of an auto referral module 518 in the remote unit 112 of FIG. 1 according to an embodiment herein. The interface view 1400 includes an automatic referral option 1402, which allows the doctor 114 to select a unit and select a user. In one embodiment, an auto referral option may be used to transmit all reports for a particular patient to another doctor 114 having another remote unit 11.

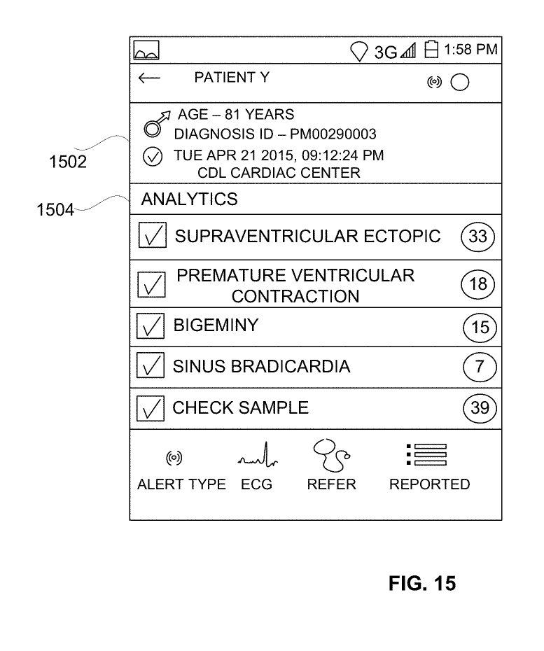

FIG. 15 illustrates a user interface view 1500 of a diagnosis summary module 512 in the remote unit 112 of FIG. 1 according to an embodiment herein. The user interface view 1500 includes a patient details view 1502 and an analytics view 1504. In one embodiment, the patient details view 1502 shows the patient 102 name, age, diagnosis ID, name of hospital, and the time the ECG diagnosis was conducted. In another embodiment, the analytics view 1504 gives details to the doctor 114 about the problems found the number of times the problem occurred during the ECG diagnosis and has a option to filter the episodes and view only the selected episodes

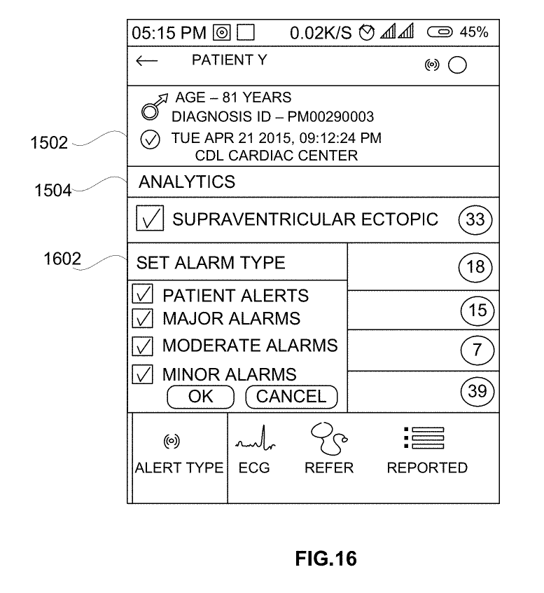

FIG. 16 illustrates a user interface view 1600 of an alert configuration module 508 in the remote unit 112 of FIG. 1 according to an embodiment herein. The user interface view 1600 includes a setting alarm type option 1602 that allows the doctor 114 to set the types of alarm notification to be received. In one embodiment, the types of alarms are patient alarms, major alarms, minor alarms, and moderate alarms.

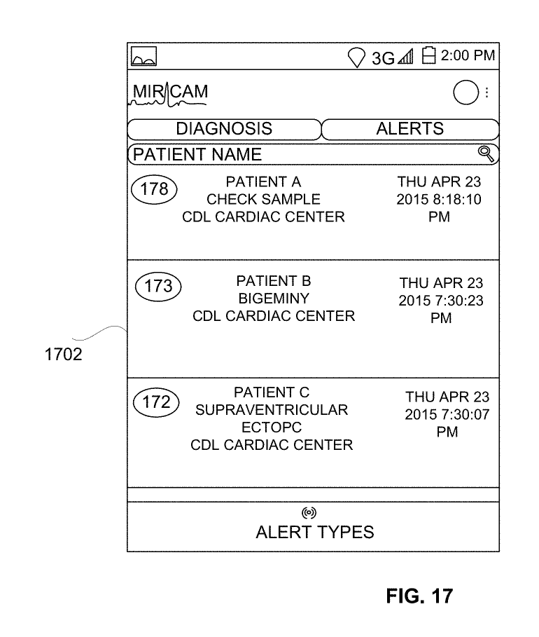

FIG. 17 illustrates a user interface view 1700 of an alert display module 516 in the remote unit 112 of FIG. 1 according to an embodiment herein. The user interface view 1700 includes an alarm list 1702 that displays the list of alarms generated from different locations and different departments. In one embodiment, the doctor 114 may filter the list by selecting the alarm type. The alert list consist of the name of the patient, the unit name, the name of the alarm generated, the name of hospital, the date that the alarm was generated, and the time that the alarm was generated.

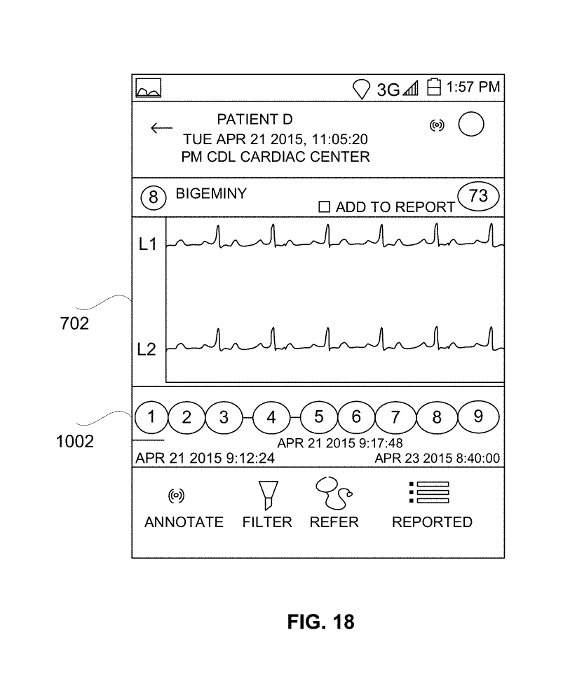

FIG. 18 illustrates a user interface view 1800 of a categorized episode timeline module 514 in the remote unit 112 of FIG. 1 according to an embodiment herein. The user interface view 1800 shows all the 12 leads recorded. Annotations of diagnoses and snapshots may be done in the ECG page. The categorized episode timeline module 514 displays various episodes detected by the base unit and indicates which category the episode relates to (e.g., patient triggered, critical, minor, major etc) based on color.

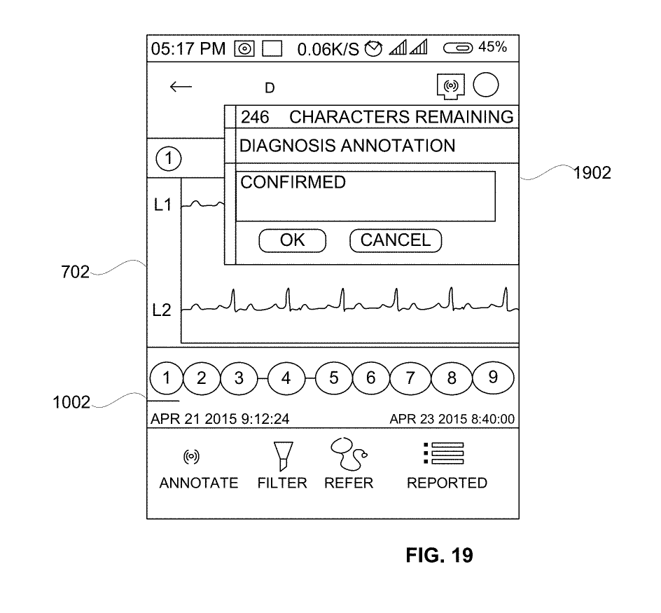

FIG. 19 illustrates a user interface view 1900 of an annotation module 520 in the remote unit 112 of FIG. 1 according to an embodiment herein. The user interface view 1900 includes a diagnosis annotation option 1902 that allows annotations of diagnosis and snapshots by the doctor 114.

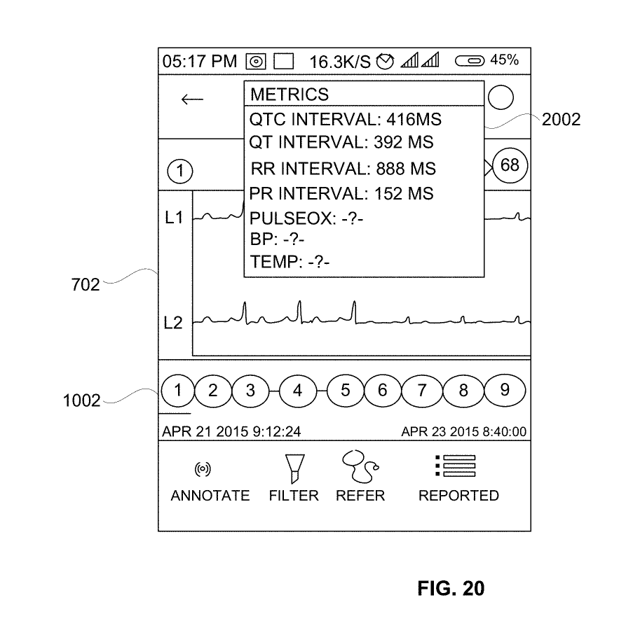

FIG. 20 illustrates a user interface view 2000 of an ECG parameters module 524 in the remote unit 112 of FIG. 1 according to an embodiment herein. The user interface view 2000 includes a heart rate label 2002 that allows the doctor 114 to view the heart rate and other parameters, such as a QTC interval, a QT interval, an RR interval, a PR interval, a pulse oximeter reading, a Blood pressure, a temperature etc.

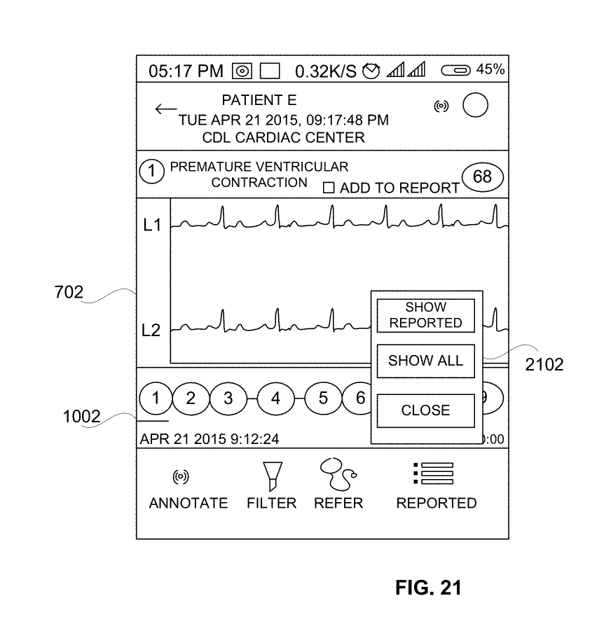

FIG. 21 illustrates a user interface view 2100 that illustrates filtering snapshots added to a report in the remote unit 112 of FIG. 1 according to an embodiment herein. The user can select any snapshot to be added into report for printing using the report append module 522 according to an embodiment.

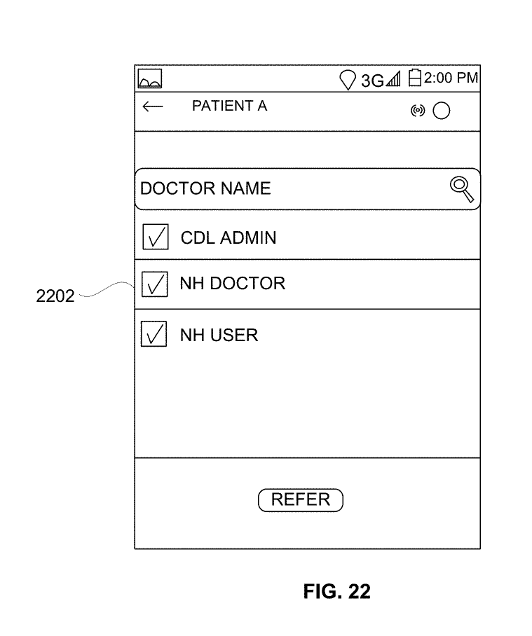

FIG. 22 illustrates a user interface view 2200 that illustrates a referral module 518 in the remote unit 112 of FIG. 1 according to an embodiment herein which involves referring a diagnosis or snapshots to other doctors according to an embodiment herein. The patient 102 name and the list of doctors for referral are displayed. In one embodiment, the doctor 112 has the option to refer the diagnosis of the patient 102 to a user, either a technician or a doctor.

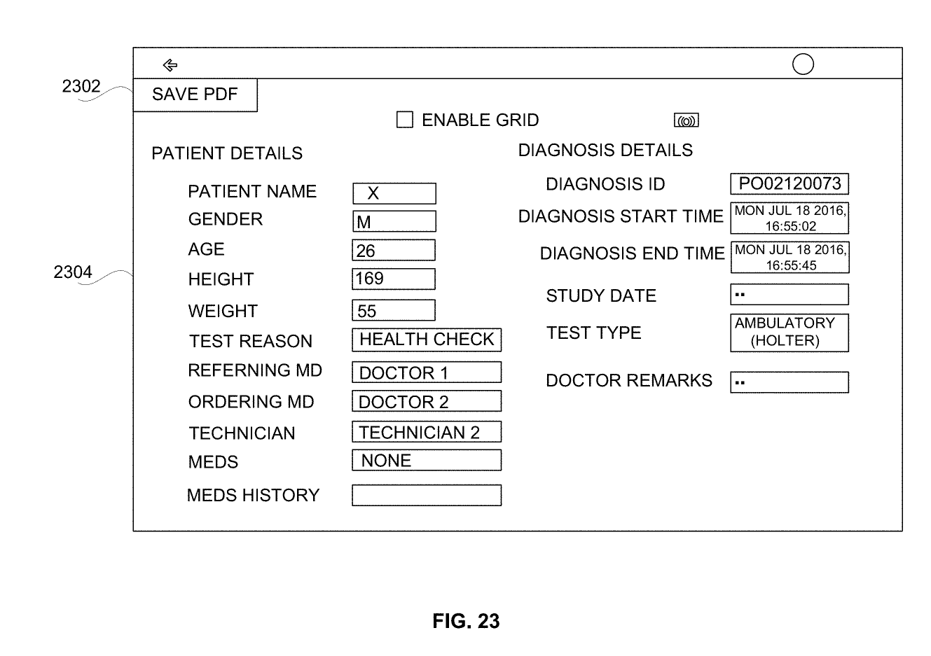

FIG. 23 illustrates a user interface view 2300 of a report generation module 526 in the remote unit 112 of FIG. 1 according to an embodiment herein. The user interface view includes a save PDF button 2302 and a report information display 2304. The save PDF button 2302 is used for generating the report and allowing the doctor 112 to save the report if ever required for future purposes. The report information display 2304 displays various details like the patient 102 information, reason for the patient 102 for conducting the diagnosis, the type of diagnosis test performed on the patient 102, the diagnosis timings, and any remarks provided by the doctor 112.

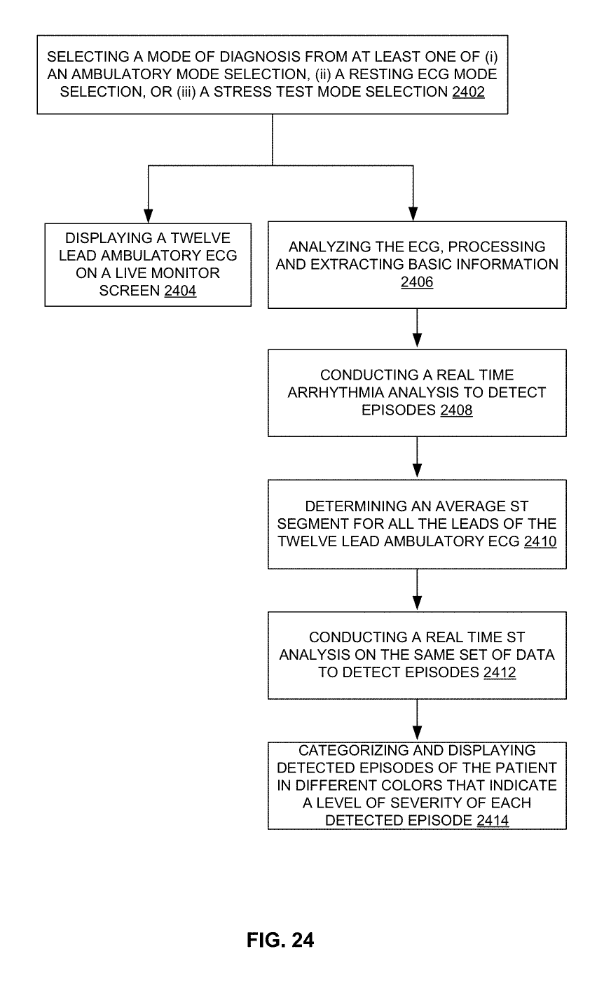

FIG. 24 is a flow diagram illustrating a method of continuous cardiac monitoring and real time episode detection that obtains live ECG data from the body worn device 104 attached to the patient 102 of FIG. 1 according to an embodiment herein. At step 2402, selection of a mode of diagnosis from at least one of (i) an ambulatory (Holter) mode selection, (ii) a resting ECG mode selection, or (iii) a stress test mode selection. At step 2404, the twelve lead ambulatory ECG is displayed on the live monitor screen during the ECG monitoring. Parallel, at step 2406, real time analysis of the ECG, processing and extraction of basic information takes place. At step 2408 real time Arrhythmia analysis is conducted to detect episodes. At step 2410, an average graph is determined for each of the ECG lead, and the average graph is displayed on the live monitor screen in ST Analysis mode. At step 2412, real time ST analysis is conducted on the same set of data during the ECG monitoring to detect episodes.

At step 2414, the detected episodes of the patient are categorized and displayed in different colors that indicate the severity of each detected episode of the patient 102 in the form of a timeline. A yellow color is displayed if the patient 102 has major problems, a blue color is displayed when the patient 102 triggers, a white color is displayed if the patient 102 has minor problems or check samples, and a red color is displayed if the patient 102 is critical.

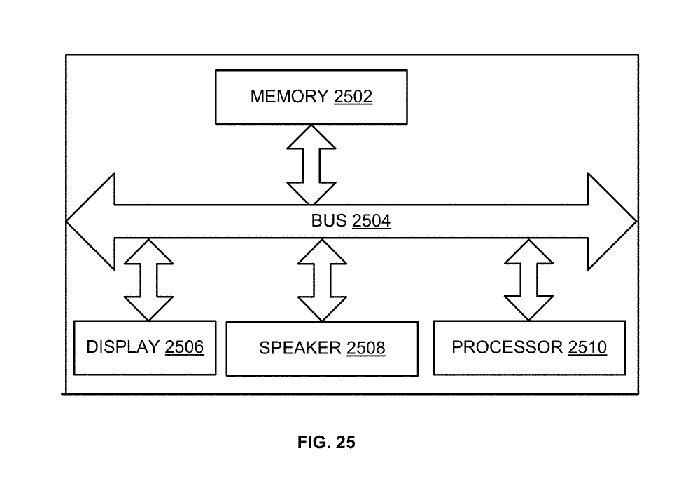

FIG. 25 illustrates an exploded view 2500 of the base unit 106 or the remote unit 112 of FIG. 1 according to an embodiment herein. The exploded view 2500 consists of a memory 2502 having a set of instructions, a bus 2504, a display 2506, a speaker 2508, and a processor 2510 capable of processing the set of instructions to perform any one or more of the methodologies herein, according to an embodiment herein. The processor 2510 may also enable digital content to be consumed in the form of video for output via one or more displays 2506 or audio for output via speaker and/or earphones 2508. The processor 2510 may also carry out the methods described herein and in accordance with the embodiments herein.

The base unit 106 provides real time analysis and communicates instant alarms to the remote unit 112 on detection of problems, thereby informing a doctor ahead of time and enhancing patient care and safety. The base unit 106 and the body worn device 104 can be used for Continuous 12 lead Ambulatory ECG Monitoring for long term (e.g., 24 hour) analysis. There are minimal chances of missing out episodes of the patient 102 through automatic instant alerts on episode detection and periodic ECG generation. The base unit 106 acts as an interactive device for a paramedic on board the ambulance, and detects multiple levels of alerts and provides expert supervision on the go in combination with the remote unit 112. The base unit 106 and the body worn device 104 may also be used for continuous monitoring and it generates instant alerts on episode detection to allow for a timely response for patients admitted in CCUs or ICUs.

The techniques provided by the embodiments herein may be implemented on an integrated circuit chip (not shown). The embodiments herein can take the form of, an entirely hardware embodiment, an entirely software embodiment or an embodiment including both hardware and software elements. The embodiments that are implemented in software include but are not limited to, firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, remote controls, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment of the base unit 106, the server 110, or the remote unit 112 for practicing the embodiments herein is depicted in FIG. 25. This schematic drawing illustrates a hardware configuration of an information handling/computer system in accordance with the embodiments herein. The system comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system can read the inventive instructions on the program storage devices and follow these instructions to execute the methodology of the embodiments herein.

The system further includes a user interface adapter 19 that connects a keyboard 15, mouse 17, speaker 24, microphone 22, and/or other user interface devices such as a touch screen device (not shown) or a remote control to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

I/We claim:

1. A continuous ECG monitoring and real time episode detection system that comprises;
a body worn device that is worn by a patient that collects eight channel ECG signals through a cable that comprises ten electrodes fixed on said patient;
a base unit, wherein said base unit is a single device that performs functions of a Holter, a resting ECG, and an event monitor and stress test, that comprises a base unit processor that executes a set of base unit modules that display, record, process, and clinically analyse live ECG data received from said body worn device, said base unit modules comprising;
a live cardiac monitor module implemented on said base unit processor that continuously displays a twelve lead ambulatory ECG on a live monitor screen;
a clinical analysis module implemented on said base unit processor that conducts a real-time arrhythmia analysis and a ST analysis on a same set of said twelve lead ambulatory ECG for a short term monitoring or a long term monitoring to detect episodes;
a ST analysis display module implemented on said base unit processor that determines an average graph for each ECG lead of said twelve lead ambulatory ECG and displays said average graph on said live monitor screen; and
an episode categorization module implemented on said base unit processor that categorizes and displays said detected episodes of said patient in different colours in the form of a timeline, depending on a level of severity of said detected episodes.

2. The continuous ECG monitoring and real time episode detection system as claimed in claim 1, wherein said base unit comprises;
a diagnosis mode selection module implemented on said base unit processor that processes a selection of a mode of diagnosis selected from the said ambulatory (Holter) mode, said resting ECG mode, or said stress test mode;
an ambulatory mode module implemented on said base unit processor that obtains said eight channel ECG signals through said cable that comprises ten electrodes to continuously monitor said patient for said long term monitoring in said ambulatory mode;
a resting ECG mode module implemented on said base unit processor that that obtains said eight channel ECG signals through said cable that comprises ten electrodes and analyses said patient for a fixed duration and immediately raises alerts if an episode is detected, in said resting ECG mode; and
a stress test mode module implemented on said base unit processor that obtains said eight channel ECG signals through said cable that comprises at least ten electrodes and implements a clinical stress protocol when said patient is running on a treadmill, in said stress test mode.

3. The continuous ECG monitoring and real time episode detection system as claimed in claim 1 comprising a remote unit, said remote unit comprising;
a remote unit processor that executes a set of remote unit modules, wherein said remote unit modules comprising;
a connection status module implemented on said remote unit processor that indicates connection status between said remote unit and said base unit;
a live ECG module implemented on said remote unit processor that displays said ECG from said base unit, when a request is made;
a categorized episode timeline module implemented on said remote unit processor that displays various episodes detected and has a filter option that filters for viewing a selected episode;
a referral module implemented on said remote unit processor that allows a doctor to refer a diagnosis or snapshots of said patient to other doctors, wherein said doctor may select another doctor by using an auto referral option that transfers said diagnosis and said snapshots of said patient to said another doctor; and
an ECG parameters module implemented on said remote unit processor that displays (i) a QTC interval, (ii) a QT interval, (iii) a RR interval, and (iv) a PR interval.

4. The continuous ECG monitoring and real time episode detection system as claimed in claim 1, wherein said body worn device comprises;
a light weight one millimeter thick coaxial cable;
a very small connector;
a power LED that indicates said body worn device battery status;
a connectivity LED that indicates a connection status with said base unit;
a patient triggered button that is pressed by said patient during an uneasy situation and an episode will be generated in said base unit and will be uploaded and received by the remote unit; and
a buzzer that beeps whenever there are disruptions in connectivity with said base unit and when there is a lead-off.

5. The continuous ECG monitoring and real time episode detection system as claimed in claim 1, wherein said clinical analysis module comprises;
a noise detection and PQRST identification module implemented on said base unit processor that detects noise based on noise threshold levels and analysis of each beat, where segments are divided between one R-peak to another R-peak looking for high and low frequency components, and a signal is logically parsed based on a amount of segments of a wave, determining where wave deviation occurs;
a reverse correction and beat selection module implemented on said base unit processor that uses features and cross referencing on other leads for beat selection, and beat characteristics are determined after a beat is detected and features of said beat are determined; and
a beat selection for ST segment module implemented on said base unit processor that selects said beat based on a ST segment.

6. The continuous cardiac monitoring and real time episode detection system of claim 1, wherein said base unit comprises;
a diagnosis settings module implemented on said base unit processor that includes system settings, diagnostic settings, and advanced settings, wherein said system settings allows said doctor to enable or disable (i) a system beep, (ii) an alarm beep, or (iii) an alarm popup, and said diagnostic settings allows said doctor to enable or disable (a) detection of said alarm conditions, (b) setting a time for periodic episode, of (c) setting a periodic count generation; and
an attendant report module implemented on said base unit processor that generates a report containing the said selected episodes detected during said ECG monitoring, where said report includes comments given by said doctor regarding said ECG diagnosis.

7. The continuous cardiac monitoring and real time episode detection system of claim 1, wherein said remote unit comprises;
an alert configuration module implemented on said remote unit processor that enables said doctor to select a type of alarm notification for receiving alerts;
an alert display module implemented on said remote unit processor that displays alerts assigned to said doctor.
an annotation module implemented on said remote unit processor that allows said doctor to annotate or add comments regarding said ECG diagnosis on which an notification will be received on the base unit and will be available to view and will be available on the report generated for printing; and
a report append module implemented on said remote unit processor that adds said snapshots of the diagnosis taken to said report.

8. A method of continuous cardiac monitoring and real time episode detection that obtains live ECG data from a body worn device attached to a patient, said method comprising;
collecting eight channel ECG signals through a cable that comprises ten electrodes fixed on said patient body;
selecting a mode of diagnosis from at least one of (i) an ambulatory mode selection, (ii) a resting ECG mode selection, or (iii) a stress test mode selection, wherein said ambulatory mode monitors a patient for a long time period, said resting ECG mode monitors ECG for a fixed duration and immediately raises alerts when an episode is detected, and said stress test mode implements a clinical stress protocolwhen said patient is running on a treadmill during an ECG monitoring;
continuously monitoring said patient during an ECG diagnosis and displaying said ECG of said patient;
conducting a real time arrhythmia analysis and a ST analysis on a same set of data during said ECG monitoring to detect episodes;
displaying a twelve lead ambulatory ECG on a live monitor screen;
determining an average graph for all the ECG leads and displaying said average graph on a live monitor screen; and
categorizing and displaying detected episodes of said patient in different colors that indicate a level of severity of each said detected episodes.

9. The method as claimed in claim 8, comprising;
allowing a doctor to enable or disable at a base unit (i) a system beep, (ii) an alarm beep, or (iii) an alarm popup; and
allowing said doctor to enable or disable at said base unit (a) detection of said alarm conditions, (b) setting a time for periodic episode, of (c) setting a periodic count generation; and
capturing one or more snapshots at said base unit, during said ECG monitoring; and
adding said one or more snapshots at said base unit, to a patient report.
